(19) United States
(12) Patent Application Publication
(10) Pub. No.: US 2026/0157711 A1
(43) Pub. Date: Jun. 11, 2026

(54) ASSESSING THE RISK OF AN UNEXPECTED MOVEMENT OF AT LEAST ONE DEVICE DURING A VASCULAR INTERVENTION

(71) Applicant: Siemens Healthineers AG, Forchheim (DE)

(72) Inventors: Kerstin Mueller, Charlottesville, VA (US); Markus Kowarschik, Erlangen (DE); Annette Birkhold, Stuttgart (DE)

(21) Appl. No.: 19/179,458

(22) Filed: Apr. 15, 2025

(30) Foreign Application Priority Data
Apr. 15, 2024 (DE) 10 2024 203 454.1

(51) Int. Cl. A61B 6/12; A61B 6/00; A61B 6/50; A61B 34/20; G06T 7/20

(52) U.S. Cl. CPC A61B 6/12; A61B 6/486; A61B 6/504; A61B 6/5217; A61B 34/20; G06T 7/20; A61B 2034/2046; G06T 2207/10116; G06T 2207/20081; G06T 2207/20084; G06T 2207/30101

(57) ABSTRACT

In order to assess the risk of an unexpected movement of at least one device during a vascular intervention, an image sequence of successive images that map a vascular structure and at least one medical device that is moving in the vascular structure is obtained. The at least one medical device is introduced into the body of a patient at an orifice. A risk characteristic value for a non-linear correlation existing or being imminent between a causal movement of the at least one medical device at an end, proximal with respect to the orifice, of the at least one medical device and a resulting movement of the at least one medical device at an end, distal with respect to the orifice, of the at least one medical device is determined. Determining the risk characteristic value includes applying a trained machine learning model to input data that contains the image sequence.

This application claims the benefit of German Patent Application No. DE 10 2024 203 454.1, filed on Apr. 15, 2024, which is hereby incorporated by reference in its entirety.

### BACKGROUND

The present embodiments relate to assessing the risk of an unexpected movement of at least one medical device during a vascular intervention, and supplying a trained machine learning model for use in such assessing.

During vascular interventions, medical devices, also referred to as medical instruments, such as vessel catheters and/or guide wires, are introduced into the blood vessels via an artificial orifice (e.g., at the hips of the patient) for minimally invasive therapy. In this connection, it is also possible (e.g., in the case of neurovascular procedures) that a plurality of such medical devices are introduced, which are also referred to as a stack or combination of devices. During the procedure, the at least one medical device is mechanically pushed by an individual carrying out the procedure or by a robot or a robotic manipulator further into the body at an end, proximal with respect to the orifice, of the at least one medical device, and this results in a movement of the at least one medical device at an end, distal with respect to the orifice, of the at least one medical device. Pulling movements and twisting movements are likewise possible. In the case of a stack of medical devices, it may be that the stack of medical devices is moved together, or that one or more devices of the stack are moved relative to one or more other devices of the stack.

During the movement of the at least one medical device in the vascular structure, the movement exerts pulling and pushing forces on the at least one medical device, and energy builds up in the at least one medical device.

When the built-up energy reduces, it is possible for the at least one medical device to slip, and this may result in a change in the state of the at least one medical device and therewith in further movements. These movements are, for example, inadvertent and uncontrolled inside the vascular structure and are also referred to as unexpected movements. First, the movements may result in the intervention being delayed, and second, also in perforation, rupture, or even in dissection of a vessel. Similar effects may occur if, for example, a guide wire or catheter is removed from the stack. The stability or the hold of the guide wire or catheter is then withdrawn from the remaining stack. This may result in it no longer remaining in place or retaining its shape but unexpectedly moving instead.

The buildup of energy may occur in parts of the at least one medical device that are not located in the immediate field of view of the individual carrying out the procedure, or are overlooked. Experienced individuals intuitively use the haptic feedback at the proximal end of the at least one medical device and/or the live fluoroscopic images when the part of the stack, which is subject to the buildup of energy, is visible in order to assess how much play has built up in the system including the medical device or stack and vessel and whether an unexpected movement of the at least one medical device exists or is imminent. However, this is unreliable (e.g., in the case of less experienced individuals or robot-guided, such as also remote-controlled, procedures).

In the publication J. Donahue et al.: “Long-term Recurrent Convolutional Networks for Visual Recognition and Description” (arXiv:1411.4389), a recurrent convolutional architecture that is suitable and may be continuously trained for extensive visual learning is described for artificial neural networks (ANNs).

In the publication X. Shi et al.: “Convolutional LSTM Network: A Machine Learning Approach for Precipitation Nowcasting” (arXiv:1506.04214), Nowcasting is regarded as a spatial-temporal sequence prediction problem in which both the input and the prediction are spatial-temporal sequences. This is achieved by the expansion of the fully connected long short-term memory (LSTM) to a convolutional LSTM (ConvLSTM).

In the publication T. N. Sainath et al.: “Convolutional, Long Short-Term Memory, fully connected Deep Neural Networks,” 2015 IEEE International Conference on Acoustics, Speech and Signal Processing (ICASSP), South Brisbane, QLD, Australia, 2015, pages 4580-4584, convolutional neural networks (CNNs), LSTMs, and deep neural networks (DNNs) are combined in a uniform architecture.

In the publication K. Zhu et al.: “LSTM enhanced by dual-attention-based encoder-decoder for daily peak load forecasting,” Electric Power Systems Research, Volume 208, 2022, 107860, a prognosis model that is based on the LSTM and is expanded by a dual encoder-decoder based on attention is described.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, the risk of an unexpected movement of at least one medical device during a vascular intervention may be more reliably assessed.

The embodiments are based on the idea of using a trained machine learning model (MLM) in order to assess from an image sequence of successive images a risk characteristic value for the risk of an unexpected correlation existing or being imminent between a causal movement and a resulting movement of the at least one medical device.

According to one aspect of the present embodiments, a computer-implemented method for assessing the risk of an unexpected movement of at least one medical device during a vascular intervention is disclosed. An image sequence of successive images is obtained. The image sequence (e.g., the successive images) map a vascular structure (e.g., of a patient), as well as at least one device that is moving in the vascular structure. The at least one medical device is introduced into a body of the patient at an orifice (e.g., an artificial orifice). A risk characteristic value is determined for an unexpected correlation existing or being imminent between a causal movement of the at least one medical device at an end, proximal with respect to the orifice, of the at least one medical device and a movement of the at least one medical device, resulting from the causal movement, at an end, distal with respect to the orifice, of the at least one medical device. Determining the risk characteristic value includes applying a trained machine learning model (MLM) to input data that contains the image sequence. For example, the risk characteristic value is determined as a function of the image sequence.

Unless stated otherwise, all acts of the computer-implemented method may be carried out by a data processing system that includes at least one data processing device. For example, the at least one data processing device is configured or adapted to execute the acts of the computer-implemented method. For this purpose, the at least one data processing device may store, for example, a computer program that includes commands that, when executed by the at least one data processing device, prompt the at least one data processing device to carry out the computer-implemented method. The computer-implemented method may also be completely or partially implemented in hardware. The expressions “data processing system” and “at least one data processing device” may be interchangeably used here and below. This also applies to corresponding expressions derived herefrom.

For the case that the at least one data processing device includes two or more data processing devices, specific steps carried out by the at least one data processing device may also be taken to be that different data processing devices carry out different steps or different parts of a step. For example, it is not necessary that each data processing device carries out the steps. In other words, the implementation of the steps may be distributed among the two or more data processing devices.

From each embodiment of the computer-implemented method, a corresponding embodiment of a method for assessing the risk of an unexpected movement of at least one medical device during a vascular intervention results. The method is not purely computer-implemented in that corresponding steps are incorporated for the generation of the image sequence.

The successive images of the image sequence may be, for example, X-ray images (e.g., may be generated by an X-ray imaging system). However, the successive images of the image sequence may also be ultrasound images generated by an ultrasound imaging system, or images generated by another imaging modality.

A trained MLM may adjust cognitive functions that humans connect with a different human understanding. For example, via training based on training data, the MLM may be capable of adapting to new circumstances and of detecting and extrapolating patterns. Another term for a trained MLM is “trained function”.

In general, the parameters of an MLM may be adapted or updated by training. For example, supervised training, semi-supervised training, unsupervised training, reinforcement learning, and/or active learning may be used. Further, representation learning, which is also referred to as feature learning, may be used. For example, the parameters of the MLMs may be iteratively adapted by a plurality of steps of the training. For example, a specific loss function that is also referred to as a cost function may be minimized during training. When training an artificial neural network (ANN), the backpropagation algorithm, for example, may be used.

An MLM may include, for example, an ANN, a support vector machine, a decision tree, and/or a Bayesian network, and/or the MLM may be based on k-means clustering, Q-learning, genetic algorithms, and/or association rules. For example, an ANN may be or include a deep neural network, a convolutional neural network, CNN, or a convolutional deep neural network. In addition, an ANN may be an adversarial network, a deep adversarial network, and/or a generative adversarial network.

The MLM used in the computer-implemented method of the present embodiments is characterized in that the MLM may process input data in the form of an image sequence. The MLM therefore uses, for example, not only the items of information contained in the individual images but also the temporal order of the images within the image sequence. This may be achieved, for example, such that the images of the image sequence are fed to the MLM one after the other and/or that the input data for each of the images of the image sequence also contains an order in the image sequence.

If the MLM is configured as an ANN, the ANN may be configured, for example, as a recurrent neural network (RNN) (e.g., as a long short-term memory (LSTM); as a recurrent convolutional neural network (RCNN)). Alternatives to the LSTM are, for example, ANNs with SRUs (e.g., single recurrent units), Jordan networks, or GRUs (gated recurrent units). Apart from an ANN, for example, Bayesian hierarchical temporal models may also be considered as an MLM, or models for support vector regression, etc.

The at least one medical device may also be referred to as at least one medical instrument. The at least one medical device may include, for example, one or more guide wires and/or one or more catheters (e.g., vessel catheters). It is also possible that the at least one medical device contains a stent or a vascular implant or the like. In general, when recording the images of the image sequence, some of the at least one medical device is positioned in the body of the patient, and another part of the at least one medical device is positioned outside of the body of the patient.

The causal movement is carried out (e.g., by a human or a robot), at the proximal end of the at least one medical device. The proximal end is situated, for example, outside of the body of the patient. The causal movement results at the distal end in the resulting movement of the at least one medical device. The proximal end is situated, for example, inside the body of the patient.

The causal movement may include, for example, pushing of the at least one medical device further into the body of the patient or pulling of the at least one medical device partially out of the body of the patient or twisting of the at least one medical device. The resulting movement is accordingly a forward movement, a backward movement, or a twisting movement of the at least one medical device. A nominal correlation, which may also be referred to as an expected correlation, exists in this case between the causal movement and the resulting movement. For example, the nominal correlation may be a linear correlation. This provides that, according to the nominal correlation during a feed of the at least one medical device by a distance S as the causal movement, the distal end of the at least one medical device similarly moves forwards by the distance S. The same applies to pulling movements or twisting movements. However, the nominal correlation does not necessarily have to be linear. For example, the nominal correlation may be supralinear, so, according to the nominal correlation (e.g., during a feed by a distance S as the causal movement), the distal end of the at least one medical device similarly moves forwards by a distance S′<S.

The unexpected correlation may be taken to be a correlation that significantly deviates from the nominal correlation. What is to be regarded as a significant deviation in this connection may be defined in an application-specific manner by the definition of one or more tolerance ranges for one or more variables that specify the correlation.

In some embodiments, a mathematical definition of the unexpected correlation or the significant deviation is not necessary. For example, the MLM may be trained in a supervised manner, with the training image sequences being annotated with the aid of human experts. The human experts have themselves performed the movement, for example, of the at least one medical device during the generation of the training image sequences. Thus, a subjectively perceived risk may be associated (e.g., with each training image sequence) by the human experts that the unexpected correlation is present. The association may be binary (e.g., “yes” versus “no”) or according to more than two values. For example, two to four values may be provided for the risk characteristic value. However, it is also possible to additionally or alternatively determine the annotations based on physical measured variables that were acquired during the movement of the at least one medical device during the generation of the training image sequences. For example, the movement amplitude or the applied force for carrying out the causal movement may be measured for this.

Once the MLM has been trained in such a way, the MLM may predict the risk characteristic value based on the image sequence. The risk characteristic value indicates how high the risk is of the unexpected correlation existing or being imminent. If such an unexpected correlation exists or is imminent, the risk of an unexpected movement of the at least one medical device (e.g., of sliding or an uncontrolled shooting-out of the at least one medical device) occurring is also increased. The risk characteristic value may be displayed or transmitted directly or in modified or processed form to the individual performing the causal movement or the corresponding robot and/or third party. As a response to this, the individual or the robot may react accordingly in order to prevent a sudden sliding or the like of the at least one medical device. Accordingly, with the aid of the present embodiments, the risk of an unexpected movement of the at least one medical device may be monitored automatically and live during a vascular intervention, and such an unexpected movement may be avoided thereby. The risk to the health of the patient is consequently reduced, and/or the risk of a delay to the vascular intervention owing, for example, to necessary corrections of the positioning of the at least one medical device or the like may be reduced.

According to at least one embodiment, the input data includes metadata. For example, the risk characteristic value is determined as a function of the metadata.

The metadata includes, for example, items of information that describe circumstances or boundary conditions during the generation of the images of the image sequence. In such embodiments, the MLM is trained to determine the risk characteristic value based on at least the image sequence and the metadata. One advantage is that fewer training datasets with corresponding training image sequences are to be processed during the training of the MLM since the additional training metadata supplies the MLM with the context to the image sequence, and thus, more efficient training is possible. Reference should be made in this connection to the fact that the advantage of more efficient training manifests itself during the training phase, the manner in which the training takes place, but also influences the application phase of the MLM, such that, for example, the corresponding metadata should also be supplied in the application phase.

According to at least one embodiment, the metadata includes patient properties of the patient. The metadata is in numerical form, for example, in this connection.

The MLM is thus provided with an important context during the generation of the image sequence, so particularly efficient training is possible. The patient properties may include age, height, weight, geometric extent of the patient, etc., as well as disease diagnoses or other anatomic properties of the patient. For example, vascular diseases, such as intracranial atherosclerotic disease (ICAD) or other arteriosclerosis diseases may be cited as disease diagnoses. Further relevant anatomical properties may be, for example, geometric properties of the vascular structure, the type of aortic arch, etc.

According to at least one embodiment, the metadata includes device properties of the at least one medical device.

The MLM is thus provided with an important context during the generation of the image sequence, so particularly efficient training is possible. The device properties may include, for example, a form or a type of the at least one medical device and/or an elasticity or a rigidity of the at least one medical device and/or include a surface quality (e.g., a surface structuring or a surface roughness) and/or a diameter of the at least one medical device and/or a working length of the at least one medical device and/or form parameters of the at least one medical device, etc.

With two or more medical devices, the device properties may include properties of the entirety of the two or more medical devices and/or properties of the individual medical devices.

According to at least one embodiment, the at least one medical device includes a first medical device and a second medical device, and the device properties include data relating to an arrangement of the first medical device and of the second medical device with respect to one another.

This may also be analogously expanded in corresponding embodiments to more than two medical devices, so the device properties include, for example, data relating to a respective arrangement of the individual medical devices.

According to at least one embodiment, the metadata includes intervention data relating to a previous course of the vascular intervention.

The MLM is thus provided with an important context during the generation of the image sequence, so particularly efficient training is possible. The previous course of the vascular intervention includes, for example, a period during which the image sequence was generated. The previous course therefore ends, for example, with the generation of a final image of the successive images of the image sequence.

According to at least one embodiment, the intervention data includes a length of a part of the at least one medical device situated in the body during a generation of the image sequence.

The length significantly influences the expected behavior of the at least one medical device and the interpretation of the image sequence. Consideration of this length as part of the metadata may therefore enable particularly efficient training.

According to at least one embodiment, the intervention data includes data relating to a feed force or pulling force applied to the at least one medical device during the generation of the image sequence, or a torque applied to the at least one medical device during the generation of the image sequence.

The feed force or pulling force or the torque significantly influences the expected behavior of the at least one medical device and the interpretation of the image sequence. Consideration of this data as part of the metadata may therefore enable particularly efficient training. The feed force or pulling force or the torque may be determined at the proximal end (e.g., using appropriate measuring devices) even during a movement of the at least one medical device. In the case of a robot or the like, which performs the movement, integrated sensors may be used.

According to at least one embodiment, the MLM includes a recurrent convolutional neural network (RCNN).

The RCNN may be taken to be an ANN that includes one or more convolutional layer(s) and one or more recurrent unit(s). First, the images of the image sequence may be efficiently processed, and second, the advantages of an RNN may be utilized thereby. In principle, the MLM may process the images of the image sequence without the information with respect to the order of the images in the image sequence. It would then also be possible to use, for example, CNNs or transformer networks that are not recurrently constructed, but as pure feed-forward networks. One advantage of using an RCNN lies, for example, in it being possible to thereby consider the temporal sequence of the images in the image sequence, and this ultimately makes it possible to supply the risk characteristic value in real time during the vascular intervention.

According to at least one embodiment, the RCNN has a convolution module that is adapted to convert the image sequence into a sequence of feature sets by applying the convolution module to the image sequence.

The convolution module includes, for example, at least one convolutional layer and/or is configured as a CNN. By applying the convolution module to the image sequence, the convolution module generates (e.g., for each image of the image sequence) exactly one feature set of the sequence of feature sets. Converting the image sequence into the sequence of feature sets may also be referred to as feature extraction from the images of the image sequence or as encoding of the images of the image sequence.

According to at least one embodiment, the RCNN has a recurrence module that is configured to generate a shared feature set by applying the recurrence module to the sequence von feature sets.

The recurrence module is, for example, an RNN (e.g., an LSTM). The convolution module and the recurrence module together may also be construed, for example, as an encoder module or feature encoder of the RCNN.

According to at least one embodiment, the RCNN has a prediction module that is configured to predict the risk characteristic value as a function of the shared feature set.

The prediction module may be a decoder module. The prediction module may be an ANN for classification. In this case, the determination of the risk characteristic value may be carried out as a binary classification or multi-class classification. Alternatively, the risk characteristic value may be predicted by regression if the prediction module is an ANN for the regression.

In embodiments in which the input data contains the metadata, for example, a supplemented feature set may be generated in that the shared feature set and the metadata are combined (e.g., concatenated or merged in some other way). The prediction module is configured to predict the risk characteristic value by applying the prediction module to the supplemented feature set. Alternatively, the feature sets of the sequence of feature sets may be combined with the metadata respectively and the shared feature set generated based on the combinations. The prediction module may then predict the risk characteristic value by applying the prediction module to the shared feature set.

In embodiments in which the input data does not contain the metadata, the prediction module may be applied, for example, to the shared feature set in order to predict the risk characteristic value.

According to at least one embodiment, a nominal correlation between the causal movement and the resulting movement is a linear correlation. The unexpected correlation between the causal movement and the resulting movement corresponds to a correlation that deviates from the nominal correlation by more than a predefined tolerance.

In order to define such a tolerance, the correlation between the causal movement and the resulting movement may be, for example, linearly approximated, and a difference between the correlation between the causal movement and its linear approximation may be determined. The correlation is then, for example, an unexpected correlation if the difference is greater than a predefined threshold value. However, other possibilities may also be used to quantify the deviation of the correlation between the causal movement and the resulting movement from the nominal correlation, and to compare the deviation with the predefined tolerance.

According to at least one embodiment, the causal movement and the resulting movement are feed movements respectively or pulling movements respectively or twisting movements respectively.

According to at least one embodiment, the at least one medical device includes one or more vessel catheters and/or one or more guide wires.

According to at least one embodiment, a user output is generated and output as a function of the risk characteristic value.

The output may reflect the risk characteristic value, for example, directly, or reflect a category of the risk characteristic value (e.g., “high risk,” “medium risk,” “low risk,” or the like). The output may take place, for example, visually and/or acoustically and/or haptically, via a corresponding output device (e.g., a display device, a loudspeaker, and/or a haptic output device). As a result, the individual carrying out the vascular intervention and/or an individual monitoring or observing the vascular intervention may be advised immediately of a possible increased risk of an unexpected movement and may react accordingly.

The computer-implemented method according to the present embodiments includes neither an introduction of the at least one medical device into the body of the patient, nor a generation of the artificial orifice (e.g., for introducing the at least one medical device into the body), nor the carrying out of the causal movement, nor other interactions with the body of the patient. This also applies in general to embodiments of the method that are not purely computer-implemented.

According to a further aspect of the present embodiments, however, a method is also disclosed that, apart from the acts of an embodiment of a computer-implemented method for assessing the risk of the unexpected movement of the at least one medical device during the vascular intervention, includes one or more of the following steps: effecting the artificial orifice for introducing the at least one medical device into the body of the patient; and/or introducing the at least one medical device into the body of the patient and into the vascular structure; and/or moving the at least one medical device in the vascular structure by effecting a corresponding causal movement at the proximal end of the at least one medical device during the generation of the image sequence.

According to a further aspect of the present embodiments, a computer-implemented training method for supplying a trained MLM for use in a computer-implemented method of the present embodiments for assessing the risk of an unexpected movement of at least one medical device during a vascular intervention is disclosed. The MLM is obtained in an untrained or partially trained state. A training image sequence of successive training images that map or simulate a vascular structure, as well as at least one device that is moving in the vascular structure, is obtained. The at least one medical device is introduced into the body of a patient at an orifice. A training risk characteristic value is determined for an unexpected correlation existing or being imminent between a causal movement of the at least one medical device at an end, proximal with respect to the orifice, of the at least one medical device and a resulting movement at an end, distal with respect to the orifice, of the at least one medical device. Determining the training risk characteristic value includes applying the MLM to training input data that includes the image sequence. A predefined loss function is evaluated as a function of the training risk characteristic value and a predefined ground truth value for the training image sequence (e.g., as a function of a difference between the training risk characteristic value and the ground truth value). The MLM is updated as a function of a result of the evaluation of the loss function.

If the MLM is an ANN, updating the MLM includes, for example, updating weights of the MLM (e.g., using a backpropagation algorithm).

The training images may be obtained, for example, by simulation of a vascular intervention and/or from actual interventions on humans or animals and/or via vascular interventions on phantom objects.

Customary regression losses or classification losses may be used as the loss function, as is also explained in the publications mentioned in the introduction.

Unless stated otherwise, all acts of the computer-implemented training method may be carried out by a further data processing system that includes at least one further data processing device. For example, the at least one further data processing device is configured or adapted to execute the acts of the computer-implemented training method. For this purpose, the at least one further data processing device may store, for example, a further computer program that includes commands that, when executed by the at least one further data processing device, prompt the at least one further data processing device to carry out the computer-implemented training method.

For the case that the at least one further data processing device includes two or more further data processing devices, specific steps carried out by the at least one further data processing device may also be taken to be that different further data processing devices carry out different steps or different parts of a step. For example, it is not necessary for each further data processing device to carry out the steps. In other words, the implementation of the steps may be distributed among the two or more further data processing devices.

From each embodiment of the computer-implemented method, a corresponding embodiment of a training method that is not purely computer-implemented results in that corresponding acts are incorporated for the generation of the training image sequence and/or of the ground truth value.

According to at least one embodiment of the computer-implemented training method, the training input data includes training metadata that includes patient properties and/or includes device properties of the at least one medical device and/or includes intervention data.

Reference is made in this regard to the explanations relating to the metadata with respect to the computer-implemented method for assessing the risk of an unexpected movement of at least one medical device.

According to at least one embodiment of the computer-implemented method for assessing the risk of an unexpected movement of at least one medical device, the MLM was or is trained using a computer-implemented training method of the present embodiments.

According to a further aspect of the present embodiments, a data processing system that is configured to carry out a computer-implemented method of the present embodiments for assessing the risk of an unexpected movement of at least one medical device is provided.

According to a further aspect of the present embodiments, an X-ray imaging system that includes an X-ray source and an X-ray detector, as well as a data processing system of the present embodiments, is provided. The X-ray imaging system may be, for example, an X-ray angiography system.

Further embodiments of the X-ray imaging system follow directly from the various embodiments of the method of the present embodiments, and vice versa. For example, individual features and corresponding explanations, as well as advantages with respect to the various embodiments relating to the method of the present embodiments, may be transferred analogously to corresponding embodiments of the X-ray imaging system. For example, the X-ray imaging system of the present embodiments is configured or programmed for carrying out a method of the present embodiments for assessing the risk of an unexpected movement of at least one medical device during a vascular intervention, or the X-ray imaging system carries out such a method.

According to a further aspect of the present embodiments, a further data processing system that is configured to carry out a computer-implemented training method of the present embodiments is provided.

According to a further aspect of the present embodiments, a computer program with commands is disclosed. When the commands are executed by a data processing system, the commands prompt the data processing system to carry out a method of the present embodiments for assessing the risk of an unexpected movement of at least one medical device.

The commands may be in the form, for example, of program code. The program code may be supplied, for example, as binary code or Assembler and/or as a source code of a programing language (e.g., C) and/or as a program script (e.g., Python).

According to a further aspect of the present embodiments, a further computer program with further commands is disclosed. When the further commands are executed by a further data processing system, the further commands prompt the further data processing system to carry out a training method of the present embodiments.

The further commands may be in the form, for example, of program code. The program code may be supplied, for example, as binary code or Assembler and/or as a source code of a programing language (e.g., C) and/or as a program script (e.g., Python).

According to a further aspect of the present embodiments, a computer-readable storage medium that stores a computer program of the present embodiments and/or a further computer program of the present embodiments is provided.

The computer program, the further computer program, and the computer-readable storage medium are computer program products respectively with the commands or the further commands.

Above and hereinafter, the solution of the present embodiments is described both in relation to the claimed systems and in relation to the claimed methods. Features, advantages, or alternative embodiments may be associated with the other claimed subject matters, and vice versa. In other words, the claims and embodiments for the systems may be improved by features that are described or claimed in connection with the respective method. In this case, the functional features of the method are implemented by physical units of the system.

Further, above and hereinafter, the solution of the present embodiments is described in relation to methods and systems for assessing the risk of an unexpected movement of at least one medical device, as well as in relation to methods and systems for supplying a trained MLM. Features, advantages, or alternative embodiments may be associated with the other claimed subject matters, and vice versa. In other words, claims and embodiments for supplying a trained MLM may be improved by features that are described or claimed in connection with the assessment of the risk of the unexpected movement. For example, the datasets used in the methods and systems may have the same properties and features as the corresponding datasets that are used in the methods and systems for supplying a trained MLM, and the trained MLMs supplied by the respective methods and systems may be used in the methods and systems for assessing the risk of the unexpected movement.

Further features and feature combinations of the present embodiments may be found in the figures and their description as well as in the claims. For example, further embodiments do not necessarily have to contain all features of one of the claims. Further embodiments may have features or feature combinations that are not mentioned in the claims.

The present embodiments will be explained in more detail below using specific example embodiments and associated schematic drawings. In the figures, same or functionally same elements may be provided with the same reference numerals. The description of same or functionally same elements is possibly not necessarily repeated with respect to different figures.

### BRIEF DESCRIPTION OF THE DRAWINGS

FIG. 1 shows a schematic representation of an example embodiment of an X-ray imaging system;

FIG. 2 shows a schematic flowchart of an example embodiment of a computer-implemented method for assessing the risk of an unexpected movement of at least one medical device during a vascular intervention;

FIG. 3 shows a schematic flowchart of a further example embodiment of a computer-implemented method for assessing the risk of an unexpected movement of at least one medical device during a vascular intervention;

FIG. 4 shows a schematic flowchart of a further example embodiment of a computer-implemented method for assessing the risk of an unexpected movement of at least one medical device during a vascular intervention;

FIG. 5 shows a schematic representation of an ANN;

FIG. 6 shows a schematic representation of a CNN; and

FIG. 7 shows a schematic representation of an RNN.

### DETAILED DESCRIPTION

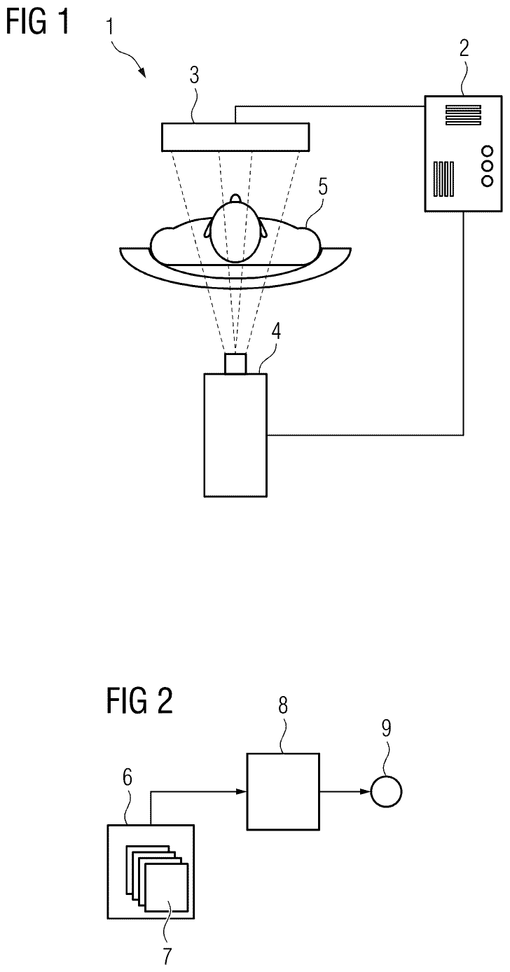

FIG. 1 schematically represents an example embodiment of an X-ray imaging system 1. The X-ray imaging system 1 has an X-ray source 4 and an X-ray detector 3, using which X-ray images 7 of a patient 5 may be generated.

The X-ray imaging system 1 may be used, for example, during a vascular intervention on a vascular structure of the patient 5 in which at least one medical device (e.g., at least one guide wire and/or at least one vessel catheter for minimally invasive procedures) that is introduced into the body of the patient 5 via an orifice are moved in the vascular structure of the patient 5 in order to bring the at least one medical device to a target position in the vascular structure. The vascular intervention takes place, for example, with X-ray support. It is possible for corresponding X-ray images 7 to be generated in respective successive frames X and be displayed on a display device of the X-ray imaging system 1. Thus, for example, an individual carrying out the treatment may observe and track, inter alia, the movement of the at least one medical device at an end, distal with respect to the orifice, of the at least one medical device, and this is also referred to as fluoroscopy. For this, the individual moves the at least one medical device at an end, proximal with respect to the orifice, of the at least one medical device, and this is also referred to as causal movement of the at least one medical device. The causal movement then leads to a resulting movement of the at least one medical device at an end, distal with respect to the orifice, of the at least one medical device.

Stacks of medical devices (e.g., catheters for minimally invasive therapy) for possible treatments, such as for aneurysm coiling, for positioning stents, for introducing embolization material, etc., are sometimes used (e.g., in the case of neurovascular procedures). In order to maneuver the catheters to the target vessel, for example, first, a guide wire may be introduced and slowly pushed into the target vessel, with small vascular branches that are referred to as perforators, and, for example, an aneurysm dome being avoided since this may result in a perforation or rupture and cause bleeding.

After the guide wire, with a displacement, the catheters, such as guide catheters, intermittent catheters, and/or microcatheters, may be introduced in order to reach the target vessel. With two catheters, reference is also made to a biaxial stack; with three catheters, reference is also made to a triaxial stack. The number of medical devices (e.g., catheters) depends, for example, on the anatomy and the location of the anomaly to be treated.

When energy builds up in the system due to friction or resistances in the vascular structure and/or the medical devices among themselves, it is possible for the at least one medical device to slip, for example, or for other undesirable and unexpected movements of the at least one medical device to occur.

In the case of X-ray imaging system 1, the individual carrying out the treatment is reliant, for example, on the fact that the individual perceives the imminence of the unexpected movement on the current X-ray image 7 and/or based on the resistance that counters a further feed of the at least one medical device. This requires considerable expertise and experience on the part of the individual carrying out the treatment.

The X-ray imaging system 1 of the present embodiments, by contrast, has a data processing system 2 that is configured to carry out a computer-implemented method for assessing the risk of an unexpected movement of at least one medical device during a vascular intervention. FIG. 2 schematically shows a flowchart of such a method.

In this connection, the data processing system 2 receives an image sequence 7 of successive images (e.g., X-ray images) that map the vascular structure as well as at least one medical device that is moving in the vascular structure. In some embodiments, the images may also be biplanar X-ray images. The at least one medical device is introduced into the body of the patient 5 at an orifice. The data processing system 2 determines a risk characteristic value 9 for an unexpected correlation existing or being imminent between the causal movement of the at least one medical device at the proximal end of the at least one medical device and the resulting movement of the at least one medical device at the distal end of the at least one medical device. For this, the data processing system 2 applies a trained MLM 8 to input data 6 that contains the image sequence 7.

As a function of the risk characteristic value 9, the data processing system 2 may control, for example, an output device to generate and output a user output. Based on this, the individual carrying out the treatment may adjust the movement of the at least one medical device accordingly in order to reduce or eliminate slack in that, for example, the individual twists or withdraws the at least one medical device or pushes the at least one medical device further forwards.

For example, the unexpected correlation between the causal movement and the resulting movement corresponds to a correlation that deviates from a nominal correlation by more than a predefined tolerance. The nominal correlation may be, for example, a linear correlation.

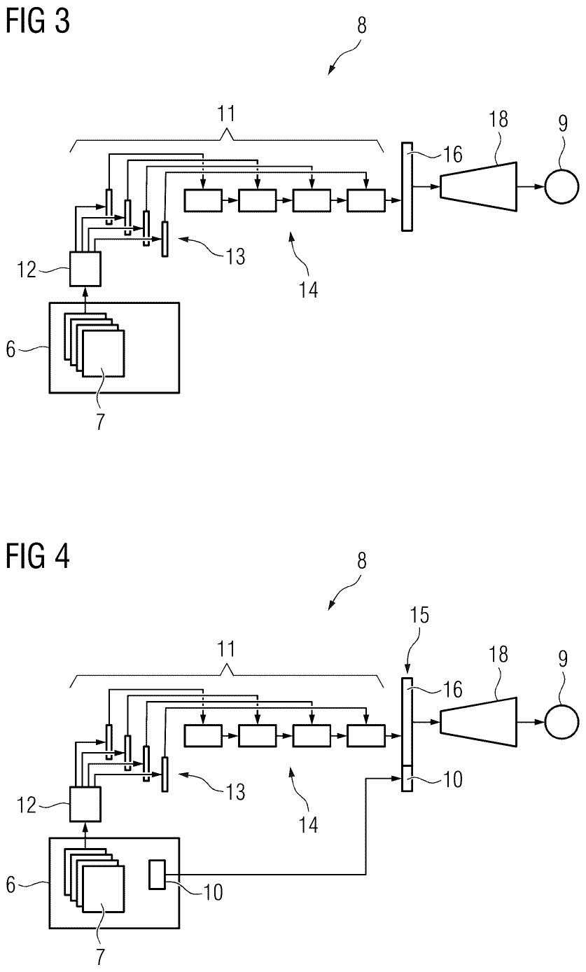

FIG. 3 shows a schematic flowchart of a further example embodiment of a computer-implemented method of the present embodiments for assessing the risk of an unexpected movement of at least one medical device during a vascular intervention, which is based, for example, on the embodiment represented in FIG. 2.

Here, the MLM 8 is configured as an RCNN that contains an encoder module 11 and a prediction module 18. The encoder module 11 includes a convolution module 12 configured as a CNN and a following recurrence module 14 that may be configured, for example, as an LSTM.

The convolution module 12 is applied to the image sequence 7 and converts the image sequence 7 thereby into a corresponding sequence of feature sets 13. These serve as input data for the prediction module 18 that generates a shared feature set 16 as the output. Applied to this is the prediction module 18 that predicts, for example, via classification or regression, the risk characteristic value.

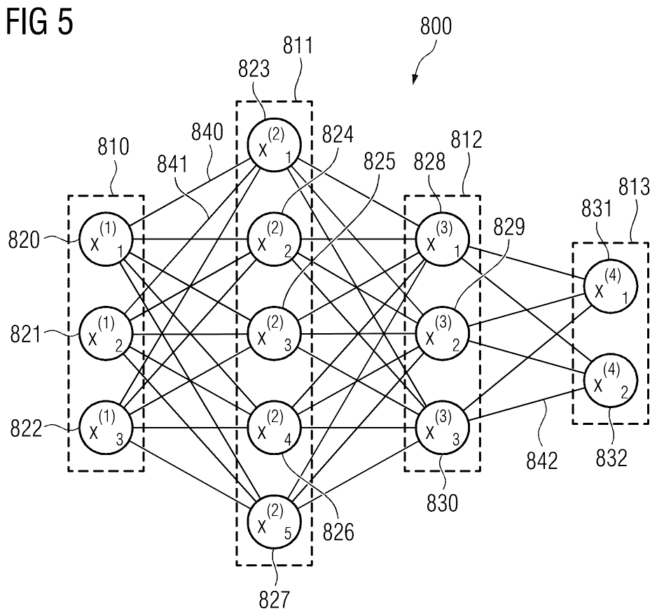

FIG. 4 shows a schematic flowchart of a further example embodiment of a computer-implemented method of the present embodiments for assessing the risk of an unexpected movement of at least one medical device during a vascular intervention that is based on the embodiment represented in FIG. 3.

The encoder module 11 is constructed as described with respect to FIG. 3. However, apart from the image sequence 7, the input data 6 in this embodiment 10 has metadata in numerical form that includes, for example, patient properties of the patient 5 and/or device properties of the at least one medical device and/or intervention data relating to a previous course of the vascular intervention. This data is merged, for example, with the shared feature set 16 (e.g., by concatenation), resulting in a supplemented feature set 15. The prediction module 18 is then applied to the supplemented feature set 15 in order to predict the risk characteristic value.

FIG. 5 shows an embodiment of an artificial neural network, ANN, 800. The ANN 800 includes nodes 820, . . . , 832 and edges 840, . . . , 842, with each edge 840, . . . , 842 being a directed connection of a first node 820, . . . , 832 to a second node 820, . . . , 832. In general, the first node 820, . . . , 832 and the second node 820, . . . , 832 are different nodes 820, . . . , 832. However, it is also possible that the first node 820, . . . , 832 and the second node 820, . . . , 832 are the same. In FIG. 5, for example, the edge 840 is a directed connection of node 820 to node 823, and the edge 842 is a directed connection of node 830 to node 832. An edge 840, . . . , 842 from a first node 820, . . . , 832 to a second node 820, . . . , 832 is also referred to as an incoming edge for the second node 820, . . . , 832 and as an outgoing edge for the first node 820, . . . , 832.

In this example, the nodes 820, . . . , 832 of the ANN 800 may be arranged in layers 810, . . . , 813, with it being possible for the layers to have an intrinsic order that is introduced between the nodes 820, . . . , 832 by the edges 840, . . . , 842. For example, the edges 840, . . . , 842 may exist only between adjacent layers of nodes. In the example shown, there is an input layer 810 that is composed only of nodes 820, . . . , 822 without incoming edges, an output layer 813 that is composed only of the nodes 831, 832 without outgoing edges, and hidden layers 811, 812 between the input layer 810 and the output layer 813. In general, the number of hidden layers 811, 812 may be selected arbitrarily. With a multilayer perceptron, MLP, this number is at least one. The number of nodes 820, . . . , 822 inside the input layer 810 refers, as a rule, to the number of input values of the artificial neural network 800, and the number of nodes 831, 832 inside the output layer 813 refers, as a rule, to the number of output values of the artificial neural network 800.

For example, a real number may be assigned as a value to each node 820, . . . , 832 of the artificial neural network 800. In this case, x⁽ⁿ⁾ᵢ designates the value of the i^{th} node 820, . . . 832 of the n^{th} layer 810, . . . , 813. The values of the nodes 820, . . . , 822 of the input layer 810 correspond to the input values of the artificial neural network 800. The values of the nodes 831, 832 of the output layer 813 correspond to the output value of the artificial neural network 800. In addition, each edge 840, . . . , 842 may have a weight that is a real number. For example, the weight is a real number within the interval [−1, 1] or within the interval [0, 1]. In this case, w^{(m,n)}_{i,j} designates the weight of the edge between the i^{th} node 820, . . . , 832 of the m^{th} layer 810, . . . , 813 and the j^{th} node 820, . . . , 832 of the n^{th} layer 810, . . . , 813. In addition, the abbreviation w⁽ⁿ⁾_{i,j} is defined for the weight w^{(n,n+1)}_{i,j}. To calculate the output values of the neural network 800, the input values, for example, are propagated by the neural network 800. For example, the values of the nodes 820, . . . , 832 of the (n+1)^{th} layer 810, . . . , 813 may be calculated based on the values of the nodes 820, . . . , 832 of the n^{th} layer 810, . . . , 813 as

$${x}_{j}^{( n + 1 )} = f ( {\sum }_{i} {x}_{i}^{( n )} {w}_{i , j}^{( n )} ) .$$

The function f is referred to therein as a transfer function or activation function. Known transfer functions are step functions, sigmoid functions (e.g., the logistical function), the generalized logistical function, the hyperbolic tangent, the arctangent function, the error function, the smoothstep function, or rectifier functions. The transfer function is primarily used for normalization. For example, the values are propagated in layers by the neural network 800, with the values of the input layer 810 being given by the input of the neural network 800. It is possible to calculate the values of the first hidden layer 811 based on the values of the input layer 810 of the neural network 800. It is possible to calculate the values of the second hidden layer 812 based on the values of the first hidden layer 811, etc.

To establish the values w^{(m,n)}_{i,j} for the edges, the neural network 800 is to be trained using training data. The training data includes, for example, training input data and training output data (referred to as tᵢ). In a training step, the neural network 800 is applied to the training-input data in order to generate calculated output data. For example, the training data and the calculated output data include a number of values that correspond to the number of nodes of the output layer. For example, a comparison between the calculated output data and the training data is used in order to recursively adapt the weights within the neural network 800 (e.g., backpropagation algorithm). For example, the weights are changed according to the following formula

$${w}_{i , j}^{′ ( n )} = {w}_{i , j}^{( n )} - γ {δ}_{j}^{( n )} {x}_{i}^{( n )} ,$$

where γ is a predefined learning rate, and the numbers δ⁽ⁿ⁾ⱼ may be recursively calculated as

$${δ}_{j}^{( n )} = ( {\sum }_{k} {δ}_{k}^{( n + 1 )} {w}_{j , k}^{( n + 1 )} ) {f}^{′} ( {x}_{i}^{( n )} {w}_{i , j}^{( n )} )$$

based on δ⁽ⁿ⁺¹⁾ⱼ if the (n+1)^{th} layer is not the output layer 813, and

$${δ}_{j}^{( n )} = ( {x}_{j}^{( n + 1 )} - {t}_{j}^{( n + 1 )} ) {f}^{′} ( {x}_{i}^{( n )} {w}_{i , j}^{( n )} ) ,$$

if the (n+1)^{th} layer is the output layer 813, where f is the first derivation of the activation function, and t⁽ⁿ⁺¹⁾ⱼ is the comparative training value for the j^{th} node of the output layer 813.

A convolutional neural network, CNN, is an ANN that uses a convolution operation in at least one of its layers instead of a general matrix multiplication. These layers are referred to as convolutional layers. For example, a convolutional layer carries out a dot product of one or more convolutional kernels with the input data of the convolutional layer, with the entries of the one or more convolutional kernels being parameters or weights that may be adapted by training. For example, the inner Frobenius product and the ReLU activation function may be used. A convolutional neural network may include additional layers (e.g., pooling layers, fully connected layers, and/or normalization layers).

By using convolutional neural networks, it is possible to process the input very efficiently since a convolution operation, which is based on different kernels, may extract different image features. Thus, by adjusting the weights of the convolutional kernel, it is possible to determine the relevant image features during the training. In addition, owing to the shared use of the weights in the convolutional kernels, fewer parameters are to be trained; this prevents an over-adjustment in the training phase and enables faster training or more layers in the network, improving the power of the network.

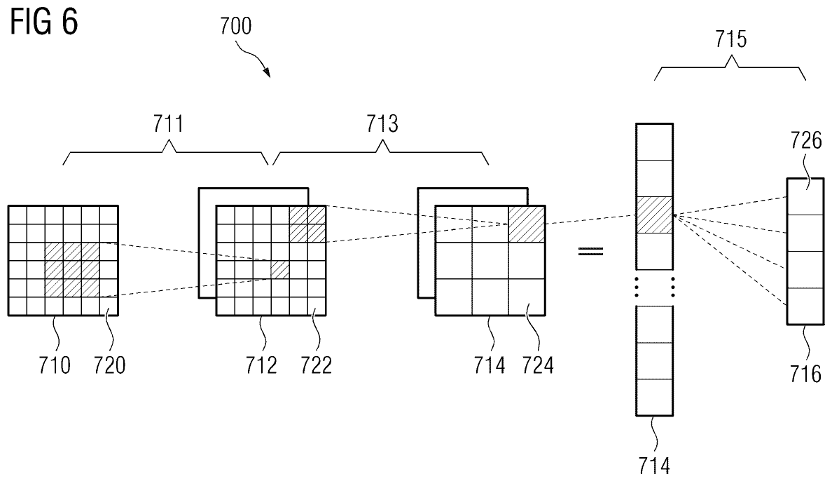

FIG. 6 shows an example embodiment of a convolutional neural network 700. In the represented embodiment, the convolutional neural network 700 includes an input node layer 710, a convolutional layer 711, a pooling layer 713, a fully connected layer 714, and an output node layer 716, as well as hidden node layers 712, 714. Alternatively, the convolutional neural network 700 may also include a plurality of convolutional layers 711, a plurality of pooling layers 713, and/or a plurality of fully connected layers 715, as well as other types of layers. The order of the layers may be selected arbitrarily; as a rule, fully connected layers 715 are used as the last layers before the output layer 716.

For example, the nodes 720, 722, 724 of a node layer 710, 712, 714 may be regarded as a d-dimensional matrix or as a d-dimensional image in a convolutional neural network 700. For example, in the two-dimensional case, the value of the node 720, 722, 724 indicated by i and j in the n^{th} node layer 710, 712, 714 may be referred to as x(n)I[i, j]. However, the arrangement of the nodes 720, 722, 724 of one node layer 710, 712, 714, as such, does not influence the calculations that are carried out within the convolutional neural network 700 since these are given solely by the structure and the weights of the edges.

A convolutional layer 711 is a connecting layer between a front node layer 710 with node values x(n−1) and a back node layer 712 with node values x(n). A convolutional layer 711 is characterized, for example, by the structure and the weights of the incoming edges that form a convolution operation based on a specific number of kernels. For example, the structure and the weights of the edges of the convolutional layer 711 are selected such that the values x(n) of the nodes 722 of the back node layer 712 are calculated as a convolution x(n)=K*x(n−1) based on the values x(n−1) of the nodes 720 of the front node layer 710, with the convolution * in the two-dimensional case being defined as

$${x}^{( n )} [ i , j ] = ( K * {x}^{( n - 1 )} ) [ i , j ] = {\sum }_{i ′} {\sum }_{j ′} K [ {i}^{′} , {j}^{′} ] \cdot {x}^{( n - 1 )} [ i - {i}^{′} , j - {j}^{′} ] .$$

In this case, the kernel K is d-dimensional matrix (e.g., in the present example, a two-dimensional matrix that, as a rule, is small compared to the number of nodes 720, 722; a 3×3 matrix or a 5×5 matrix). This provides, for example, that the weights of the edges in the convolutional layer 711 are not independent but are selected such that the weights produce the convolution equation. For example, for one kernel that is a 3×3 matrix, only 9 independent weights result, with each entry in the kernel matrix corresponding to an independent weight, irrespective of the number of nodes 720, 722 in the front node layer 710 and the back node layer 712. In general, convolutional neural networks 700 use node layers 710, 712, 714 with a large number of channels (e.g., owing to the use of a large number of kernels in the convolutional layers 711). In these cases, the node layers m be regarded as (d+1)-dimensional matrices, with the first dimension indicating the channels. The effect of a convolutional layer 711 is then defined in a two-dimensional example as

$${x}_{b}^{( n )} [ i , j ] = {\sum }_{a} ( {K}_{a , b} * {x}_{a}^{( n - 1 )} [ i , j ] = {\sum }_{a} {\sum }_{i ′} {\sum }_{j ′} {K}_{a , b} [ {i}^{′} , {j}^{′} ] \cdot {x}_{a}^{( n - 1 )} [ i - {i}^{′} , j - {j}^{′} ] ,$$

where

$${x}_{a}^{( n )}$$

corresponds to the a^{th} channel of the preceding node layer 710,

$${x}_{b}^{( n )}$$

corresponds to the b^{th} channel of the subsequent node layer 712, and K_{a,b} corresponds to one of the kernels. If a convolutional layer 711 acts on a preceding node layer 710 with A-channels and outputs a subsequent node layer 712 with B-channels, A, ·B-independent d-dimensional kernels K_{a,b} are produced.

In general, activation functions may be used in convolutional neural networks 700. In this embodiment, ReLU (rectified linear unit) is used, where R(z)=max(0, z), so the effect of the convolutional layer 711 in the two-dimensional example is

$${x}_{b}^{( n )} [ i , j ] = R ( {\sum }_{a} ( {K}_{a , b} * {x}_{a}^{( n - 1 )} [ i , j ] ) = R ( {\sum }_{a} {\sum }_{i ′} {\sum }_{j ′} {K}_{a , b} [ {i}^{′} , {j}^{′} ] \cdot {x}_{a}^{( n - 1 )} [ i - {i}^{′} , j - {j}^{′} ] )$$

It is also possible to use other activation functions (e.g., Exponential Linear Unit (ELU), LeakyReLU, Sigmoid, Tanh, or Softmax).

In the embodiment shown, the input layer 710 includes 36 nodes 720 that are arranged in a two-dimensional 6×6 matrix. The first hidden node layer 712 includes 72 nodes 722 that are arranged as two-dimensional 6×6 matrices, with each of the two matrices being the result of a convolution of the values of the input layer with a 3×3 kernel within the convolutional layer 711. In a manner equivalent to this, the nodes 722 of the first hidden node layer 712 may be interpreted as a three-dimensional 2×6×6 matrix, with the first dimension corresponding to the channel dimension.

One advantage of using convolutional layers 711 consists in that a spatial local correlation of the input data may be utilized in that a local connectivity pattern is enforced between the nodes of adjacent layers (e.g., in that each node is connected to only a small region of the nodes of the preceding layer).

A pooling layer 713 is a connecting layer between a preceding node layer 712 with node values x(n−1) and a subsequent node layer 714 with node values x(n). A pooling layer 713 may be characterized, for example, by the structure and the weights of the edges and the activation function, which form a pooling operation based on a non-linear pooling function f. For example, in the two-dimensional case, the values x(n) of the nodes 724 of the subsequent node layer 714 may be calculated based on the values x(n−1) of the nodes 722 of the anterior node layer 712 as follows

$${x}_{b}^{( n )} [ i , j ] = f ( {x}_{b}^{( n - 1 )} [ {id}_{1} , {jd}_{2} ] , … , {x}_{b}^{( n - 1 )} [ ( i + 1 ) {d}_{1} - 1 , ( j + 1 ) {d}_{2} - 1 ] ) .$$

In other words, the use of a pooling layer 713 may reduce the number of nodes 722, 724 in that a number d1-d2 of adjacent nodes 722 in the preceding node layer 712 is replaced by a single node 722 in the subsequent node layer 714, which is calculated as a function of the values of the number of adjacent nodes. The pooling function f may be, for example, the max function, the mean, or the L2 norm. For example, with a pooling layer 713, the weights of the incoming edges are fixed and are not changed by the training.

The advantage of using a pooling layer 713 is that the number of nodes 722, 724 and the number of parameters are reduced. This results in a reduction in the calculation effort in the network and in control of the overadjustment.

In the embodiment shown, the pooling layer 713 is a max-pooling layer in which four adjacent nodes are replaced by just one node, with the value being the maximum of the values of the four adjacent nodes. The max-pooling is applied to each d-dimensional matrix of the previous layer. In this embodiment, the max-pooling is applied to each of the two-dimensional matrices, whereby the number of nodes is reduced from 72 to 18.

In general, the last layers of a convolutional neural network 700 may be fully connected layers 715. A fully connected layer 715 is a connecting layer between a preceding node layer 714 and a subsequent node layer 716. A fully connected layer 713 may be characterized in that the majority (e.g., all) of edges between the nodes 714 of the preceding node layer 714 and the nodes 716 of the subsequent node layer are present, and it is possible for the weight of each of these edges to be individually adapted.

In this embodiment, the nodes 724 of the preceding node layer 714 of the fully connected layer 715 are represented as two-dimensional matrices and also as non-contiguous nodes that are displayed as a line of nodes, with the number of nodes being reduced for improved representation. This procedure is also referred to as flattening. In this embodiment, the number of nodes 726 in the subsequent node layer 716 of the fully connected layer 715 is less than the number of nodes 724 in the preceding node layer 714. Alternatively, the number of nodes 726 may also be the same or greater.

In addition, in this embodiment, the softmax activation function is used within the fully connected layer 715. Applying the softmax function provides that the sum of the values of all nodes 726 of the output layer 716 is 1, and all values of all nodes 726 of the output layer 716 are real numbers between 0 and 1. For example, when the convolutional neural network 700 is used for categorization of input data, the values of the output layer 716 may be interpreted as the probability that the input data falls into one of the different categories.

For example, convolutional neural networks 700 may be trained based on the backpropagation algorithm. To prevent an overadjustment, methods of regularization, for example, omitting nodes 720, . . . , 724, stochastic pooling, the use of artificial data, weight decay based on the L1 or L2 norm, or max-norm limitations may be used.

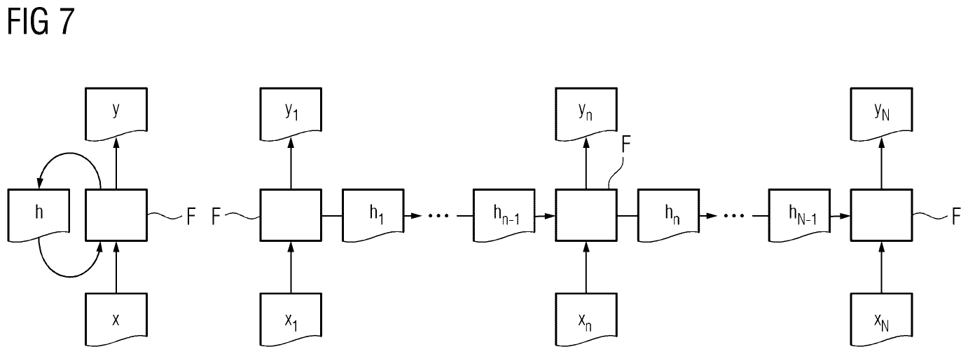

FIG. 7 shows a recurrent MLM F (e.g., a recurrent neural network (RNN)). A recurrent MLM F is a machine learning model having output that depends not only on the current input value and the parameters of the MLM adapted by the training process, but also on a hidden state vector, with the hidden state vector being based on earlier inputs that were used for the recurrent MLM F. For example, the recurrent MLM F may include additional storage states or additional structures that incorporate time delays or include feedback loops.

An RNN may be described as an ANN in which the connections between the nodes form a directed graph along a time sequence. For example, an RNN may be interpreted as a directed acyclical graph. For example, the RNN may be an RNN with finite impulses (e.g., finite impulse RNN) or an RNN with infinite impulses (e.g., infinite impulse RNN). A finite impulse RNN may be unrolled and replaced by a pure feedforward ANN, whereas an infinite impulse RNN cannot be unrolled and replaced by a pure feedforward ANN. Training of an RNN may be based, for example, on the backpropagation through time (BPTT) algorithm, on the real-time recurrent learning (RTRL) algorithm, and/or on genetic algorithms.

FIG. 7 schematically shows the structure of a recurrent MLM F on the left in a recurrent representation and on the right in an unfolded representation. As an input, the recurrent MLM F receives a plurality of input datasets x₁, . . . , x_{N} and generates a corresponding number of output datasets y₁, . . . , y_{N}. Further, the output is dependent on what is known as a hidden vector h₁, . . . , h_{N}, which implicitly includes items of information about input datasets that were previously used as an input for the recurrent MLM F. Using these hidden vectors h₁, . . . , h_{N} makes it possible to use the sequentiality of the input datasets. In a single processing step, the recurrent MLM F receives the hidden vector hₙ₋₁, created in the previous step, and an input dataset xₙ as the input. In this step, the recurrent MLM F generates an updated hidden vector hₙ and an output dataset yₙ as the output. In other words, in a processing step, the following is calculated:

$$( {y}_{n} , {h}_{n} ) = F ( {x}_{n} , {h}_{n - 1} ) .$$

Alternatively, by dividing the recurrent MLM F into a part F, that calculates the output data and a part Fₕ that calculates the hidden vector, in a processing step, the following is calculated:

$$\begin{matrix}{y}_{n} = {F}_{y} ( {x}_{n} , {h}_{n - 1} ) , \\ {h}_{n} = {F}_{h} ( {x}_{n} , {h}_{n - 1} ) .\end{matrix}$$

For the initial processing step n=1, hₙ₋₁=h₀ may be randomly selected, or all entries may be set to zero. The parameters of the recurrent MLM F, which were previously trained based on training datasets, do not change between the various processing steps. For example, the output data and the hidden vector of a processing step may depend on all previous input datasets that were used in the preceding steps:

$$\begin{matrix}{y}_{n} = {F}_{y} ( {x}_{n} , {F}_{h} ( {x}_{n - 1} , {h}_{n - 1} ) ) , \\ {h}_{n} = {F}_{h} ( {x}_{n} , {F}_{h} ( {x}_{n - 1} , {h}_{n - 1} ) ) .\end{matrix}$$

Independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A computer-implemented method for assessing a risk of an unexpected movement of at least one medical device during a vascular intervention, the computer-implemented method comprising:
obtaining an image sequence of successive images that map a vascular structure and at least one medical device that is moving in the vascular structure, wherein the at least one medical device is introduced into the body of a patient at an orifice; and
determining a risk characteristic value for an unexpected correlation existing or being imminent between a causal movement of the at least one medical device at an end, proximal with respect to the orifice, of the at least one medical device and a resulting movement of the at least one medical device at an end, distal with respect to the orifice, of the at least one medical device,
wherein determining the risk characteristic value includes applying a trained machine learning model to input data that contains the image sequence.

2. The computer-implemented method of claim 1, wherein the input data includes metadata that includes patient properties of the patient, device properties of the at least one medical device, intervention data relating to a previous course of the vascular intervention, or any combination thereof.

3. The computer-implemented method of claim 2, wherein the intervention data includes a length of a part, located in the body during generation of the image sequence, of the at least one medical device, data relating to a feed force or pulling force applied to the at least one medical device during generation of the image sequence or a torque applied to the at least one medical device during generation of the image sequence, or a combination thereof.

4. The computer-implemented method of claim 2, wherein:
the device properties include an elasticity, a rigidity, a surface quality, or any combination thereof of the at least one medical device;
the at least one medical device includes a first medical device and a second medical device, and the device properties include data relating to an arrangement of the first medical device and of the second medical device with respect to one another; or
any combination thereof.

5. The computer-implemented method of claim 2, wherein the trained machine learning model includes a recurrent convolutional neural network.

6. The computer-implemented method of claim 5, further comprising:
converting the image sequence into a sequence of feature sets, the converting comprising applying a convolution module of the recurrent convolutional neural network to the image sequence; and
generating a shared feature set, the generating comprising applying a recurrence module of the recurrent convolutional neural network to the sequence of feature sets,
wherein determining the risk characteristic value comprises predicting the risk characteristic value as a function of the shared feature set by a prediction module of the recurrent convolutional neural network.

7. The computer-implemented method of claim 6, further comprising generating a supplemented feature set, the generating of the supplemented feature set comprising combining the shared feature set and the metadata,
wherein predicting the risk characteristic value comprises applying the prediction module to the supplemented feature set.

8. The computer-implemented method of claim 1, wherein a nominal correlation between the causal movement and the resulting movement is a linear correlation, and
wherein the unexpected correlation between the causal movement and the resulting movement corresponds to a correlation that deviates from the nominal correlation by more than a predefined tolerance.

9. The computer-implemented method of claim 1, wherein the at least one medical device includes one or more vessel catheters, one or more guide wires, or the one or more vessel catheters and the one or more guide wires.

10. The computer-implemented method of claim 1, further comprising:
generating a user output; and
outputting the user output as a function of the risk characteristic value.

11. A computer-implemented training method for supplying a trained machine learning model for use in a computer-implemented method for assessing a risk of an unexpected movement of at least one medical device during a vascular intervention, the computer-implemented training method comprising:
obtaining a machine learning model in an untrained or partially trained state;
obtaining a training image sequence of successive training images that map or simulate a vascular structure, as well as at least one device that is moving in the vascular structure, wherein the at least one medical device is introduced into the body of a patient at an orifice;
determining a training risk characteristic value for an unexpected correlation existing or being imminent between a causal movement of the at least one medical device at an end, proximal with respect to the orifice, of the at least one medical device and a resulting movement at an end, distal with respect to the orifice, of the at least one medical device, wherein determining the training risk characteristic value comprises applying the machine learning model to training input data that contains the image sequence;
evaluating a predefined loss function as a function of the training risk characteristic value and a predefined ground truth value for the training image sequence; and
updating the machine learning model as a function of a result of the evaluating of the predefined loss function.

12. The computer-implemented training method of claim 11, wherein the training input data includes training metadata that includes patient properties, device properties of the at least one medical device, intervention data, or any combination thereof.

13. The computer-implemented method of claim 1, further comprising training the machine learning model, the training of the machine learning model comprising:
obtaining the machine learning model in an untrained or partially trained state;
obtaining a training image sequence of successive training images that map or simulate a vascular structure, as well as at least one device that is moving in the vascular structure;
determining a training risk characteristic value for the unexpected correlation existing or being imminent between a causal movement of the at least one medical device at the end, proximal with respect to the orifice, of the at least one medical device and a resulting movement at the end, distal with respect to the orifice, of the at least one medical device, wherein determining the training risk characteristic value comprises applying the machine learning model to training input data that contains the image sequence;
evaluating a predefined loss function as a function of the training risk characteristic value and a predefined ground truth value for the training image sequence; and
updating the machine learning model as a function of a result of the evaluating of the predefined loss function.

14. A data processing system comprising:
a processor configured to assess a risk of an unexpected movement of at least one medical device during a vascular intervention, the processor being configured to assess the risk of the unexpected movement of the at least one medical device during the vascular intervention comprising the processor being configured to:
obtain an image sequence of successive images that map a vascular structure and at least one medical device that is moving in the vascular structure, wherein the at least one medical device is introduced into the body of a patient at an orifice; and
determine a risk characteristic value for an unexpected correlation existing or being imminent between a movement of the at least one medical device at an end, proximal with respect to the orifice, of the at least one medical device and a resulting movement of the at least one medical device at an end, distal with respect to the orifice, of the at least one medical device,
wherein the determination of the risk characteristic value includes application of a trained machine learning model to input data that contains the image sequence.
